(19) United States
(12) Patent Application Publication
(10) Pub. No.: US 2011/0301099 A1
(43) Pub. Date: Dec. 8, 2011

(54) Method for enhancing penetration of lipid insoluble medicaments through cell membranes using small S-Allyl-containing molecules with phospholipid permeability as carrier molecules

(76) Inventor: Madalene Choon Ying Heng, Northbridge, CA (US)

(21) Appl. No.: 12/802,354

(22) Filed: Jun. 4, 2010

(51) Int. Cl. A61K 38/12; A61K 31/12; A61K 31/522; A61K 31/52; A61K 31/135; A61P 31/00; A61K 31/4164; A61K 31/496; A61K 31/7036; A61P 31/12; A61P 31/10; A61K 31/167; A61K 31/343

(52) U.S. Cl. 514/21.1; 514/626; 514/679; 514/263.38; 514/263.4; 514/657; 514/462; 514/396; 514/254.07; 514/39

(57) ABSTRACT

A method and composition that allows for the penetration of lipid insoluble medicaments through cell membranes using small S-Allyl-containing molecules with phospholipid permeable molecules as carrier molecules. S-Allyl-containing compounds can include alliin, allicin, Allium sativum extract and DMSO. Lipid insoluble medicaments can include 1% xylocaine, curcumin, acyclovir, valacyclovir, famciclovir, terbenafin, griseofulvin, clotrimazole, ketoconazole, clindamycin, neomycin, polysporin, bactricin, among others. The lipid insoluble medicaments are mixed with the phospholipid permeable molecules and painted on the skin using a swab or other suitable tool, thereby allowing for penetration of the medicament and providing relief from pain potentially caused by a subsequent injection. The composition can also be administered through an inhaler or directly into mucous membranes. If appropriately packaged and given intravenously, S-allyl molecules may also be used to enhance the penetration of non-lipid soluble molecules, including curcumin, through cell membranes in solid organs, such as the liver, pancreas, kidneys, uterus, prostrate, bone, joints and brain.

### BACKGROUND OF THE INVENTION

1. Field of the Invention

The present invention relates to chemical compositions and their use and specifically toward a method for enhancing the penetration of lipid insoluble medicaments through cell membranes using small S-allyl-containing molecules with phospholipid permeability as carrier molecules.

2. Description of the Prior Art

Garlic is a spice with a varied number of properties, including anti-carcinogenic, cardio-protective, anti-viral, anti-microbial and anti-fungal properties. The active ingredient is alliin (S-allyl-cysteine sulfoxide), which is metabolized to allicin (N-acetyl-S allyl-cysteine) by an enzyme released when its membranes are crushed. Other metabolites include allyl mercaptan, diallyl disulfide, diallyl sulfide, diallyl sulfoxide, diallyl sulfone and methyl sulfide.

Garlic has been thought to bring about its anti-carcinogenic effect through a number of mechanisms, such as the scavenging of free radicals by increasing free radical quenchers, such as glutathione and catalases, prevention of chromosomal damage and increasing DNA repair mechanisms (Kahnum F, Anilakumar K R, Viswanathan K R, Anticarcinogenic properties of garlic: a review. Crit Rev Food Sci Nutr 2004; 44:479-488), as well as by inducing apoptosis (cell death) of cancer cells (Miron T, Wilchek M, Sharp et al. Allicin inhibits cell growth and induces apoptosis through the mitochondrial pathway in HL-60 and U937 cells. J Nutr Biochem 2008; 19:524-535; Park S Y, Cho S J, Kwon H C et al. Caspase independent cell death by allicin in human epithelial carcinoma cells: involvement of PKA. Cancer Lett 2005; 224:123-132; Zhang Z M, Zhong M, Gao H Q et al. Inducing apoptosis and upregulation of Bax and Fas ligand expression by allicin in hepatocellular carcinoma in Balb/c nude mice. Chin Med J/English 2006; 119:422-425).

Cardio-protective properties have been linked to vasoactivity of garlic, which has been linked to mediation by hydrogen sulfide (Benevides G A, Squadrito G L, Mills R W et al. Hydrogen sulfide mediates the vasoactivity of garlic. Proc Natl Acad Sci USA 2007; 104: 17907-17908). Additionally, its hypocholesterolemic effect has been linked to suppression of HMG-reductase expression and inhibition of cholesterol synthesis (Liu L, Yeh Y Y. Inhibition of cholesterol synthesis by organophosphate compounds derived from garlic. Lipids 2000; 35: 197-203; Rai S K, Sharma M, Tiwari M. Inhibitory effect of a novel diallylsulfide analogs on HMG-CoA reductase expression is hypercholesterolemic rats: CREB as potential upstream target, PMID 19323964 (Pub Med indexed on Medline).

Allicin has been found to have anti-viral properties (Weber N D, Anderson D O, North J A et al. In vitro-virucidal effects of Allium sativum (garlic) extract and compounds. Planta Med 1992; 58:417-23) as well as anti-fungal properties (Davis S R. On overview of the antifungal properties of allicin and its breakdown products—the possibility of a safe and effective antifungal prophylactic. Mycoses 2005; 48:95-100). Moreover, allicin has been shown to enhance the oxidative damage of Amphotericin B against Candida albicans (An M, Shen H, Cao Y et al. Allicin enhances the oxidative damage of Amphotericin B against Candida albicans. Int J Antimicrob Agents 2009: 33:258-263). Additionally, the anti-fungal properties of allicin have also been linked to increased intracellular ergosterol trafficking (Ogita A, Fujita K, Tanaka T. Enhancement of fungicidal activity of Amphotericin B by allicin: effects of intracellular ergosterol trafficking. Planta Med 2009; 75:222-226).

The ability of allicin to permeate phospholipid membranes has been found by the instant inventor and has been confirmed by others (Tsuchiya H, Nagayama M. Garlic allyl derivatives interact with membrane lipids to modify membrane fluidity. J Biomed Sci 2008; 15:653-660). More recently, garlic allyl derivatives have been reported to interact with membrane lipids to modify the membrane fluidity (Miron T, Rabinkov A, Mirelman D et al. The mode of action of allicin: its ready permeability through phospholipids membranes contribute to its biological activity. Biochim Biophys Acta 2000; 1463:20-30), thus lending further support to previous observations. However, the notion of using the lipid permeability properties of the S-allyl molecules in garlic and DMSO to serve as carrier molecules to transport non-lipid soluble molecules across cell membranes, has not hitherto been reported.

It is the primary object of the instant invention to provide a method for using the permeability properties of the S-allyl molecules in garlic and DMSO to serve as carrier molecules to transport non-lipid soluble molecules across cell membranes.

### SUMMARY OF THE INVENTION

The preferred embodiment is a composition providing increased penetration of lipid insoluble medicaments through cell membranes comprising: an S-Allyl-containing molecule; and a lipid insoluble medicament.

The above embodiment can be further modified by defining that the composition is put into a gel medium.

The above embodiment can be further modified by defining that the said S-Allyl-containing molecule is taken from the following group: alliin, allicin, Allium sativum extract, DMSO.

The above embodiment can be further modified by defining that the lipid insoluble medicament is taken from the following group: xylocaine, curcumin, acyclovir, valacyclovir, famciclovir, terbenafin, griseofulvin, clotrimazole, ketoconazole, clindamycin, neomycin, polysporin, bactricin.

The above embodiment can be further modified by defining that the composition is housed in an inhaler.

The above embodiment can be further modified by defining that the composition is applied directly to mucous membranes.

A second embodiment teaches a method of penetrating cell membranes with lipid insoluble medicaments comprising the steps of: preparing a composition comprising an S-Allyl-containing molecule; and a lipid insoluble medicament; and applying said composition to said site of said cell membranes with a swab or other application device.

The above embodiment can be further modified by defining that the there is a further step of injecting said lipid insoluble medicament into said site of said cell membranes.

The above embodiment can be further modified by defining that the composition is put into a gel medium.

The above embodiment can be further modified by defining that the S-Allyl-containing molecule is taken from the following group: alliin, allicin, Allium sativum extract, DMSO.

The above embodiment can be further modified by defining that the lipid insoluble medicament is taken from the following group: xylocaine, curcumin, acyclovir, valacyclovir, famciclovir, terbenafin, griseofulvin, clotrimazole, ketoconazole, clindamycin, neomycin, polysporin, bactricin.

A second alternate embodiment teaches a method of penetrating cell membranes with lipid insoluble medicaments comprising the steps of: preparing a composition comprising an S-Allyl-containing molecule; and a lipid insoluble medicament; and administering said composition through an inhaler.

The above embodiment can be further modified by defining that the S-Allyl-containing molecule is taken from the following group: alliin, allicin, Allium sativum extract, DMSO.

The above method can be further modified by defining that the lipid insoluble medicament is taken from the following group: xylocaine, curcumin, acyclovir, valacyclovir, famciclovir, terbenafin, griseofulvin, clotrimazole, ketoconazole, clindamycin, neomycin, polysporin, bactricin.

A third alternate embodiment teaches a method of penetrating cell membranes with lipid insoluble medicaments comprising the steps of: preparing a composition comprising an S-Allyl-containing molecule; and a lipid insoluble medicament; and administering said composition directly to a mucous membrane.

The above embodiment can be further modified by defining that the S-Allyl-containing molecule is taken from the following group: alliin, allicin, Allium sativum extract, DMSO.

The above embodiment can be further modified by defining that the lipid insoluble medicament is taken from the following group: xylocaine, curcumin, acyclovir, valacyclovir, famciclovir, terbenafin, griseofulvin, clotrimazole, ketoconazole, clindamycin, neomycin, polysporin, bactricin.

### DETAILED DESCRIPTION OF A PREFERRED EMBODIMENT

Many medicaments, including xylocaine, a local anesthetic, are incapable of penetration through undamaged cell membranes such as the stratum corneum. It is therefore not currently feasible to deliver topical xylocaine directly the skin without a needle to penetrate the stratum corneum.

We have discovered that by using alliin and/or allicin in Allium sativum (garlic) extract, we can enhance the penetration of topical 1% xylocaine through the stratum corneum such that numbing of the skin is produced by merely painting a mixture of Allium sativum extract and 1% xylocaine in a gel medium on the skin. Following the contact with this mixture, the skin is numbed so that a subsequent injection of 1% xylocaine delivered into the subcutaneous tissue causes very little discomfort to the patient. In the same patient, 1% xylocaine injected subcutaneously in control sites not previously treated with Allium sativum/1% xylocaine extract produced pain and discomfort to the patient.

Since alliin (S-allyl cysteine sulfoxide) and allicin (N-acetyl-S allyl-cysteine or diallylthiosulfate) in Allium sativum (garlic) extract have the capability to dissolve in lipids, this may contribute to its ability to penetrate cell membranes (Heng, unpublished data, 1982), which are composed of a two layers of phospholipids. However, since 1% xylocaine is not lipid soluble, the local anesthetic does not have the capability of penetrating the lipid-containing cell membranes, including the stratum corneum. Using a mixture of 1% xylocaine and Allium sativum extract containing both alliin and allicin, in a gel medium for easy application to the skin with a swab, we are able to use the Allium sativum extract as a carrier molecule to enhance the penetration of a non-lipid soluble medicament, such as 1% xylocaine, rapidly (within one minute) through lipid cell membranes, including the stratum corneum (Heng M C Y; unpublished data, 2010). We have found that other S-allyl containing small molecules such as dimethylsulfoxide (DMSO) can also function in the same way (Heng, M C Y, unpublished data, 2010).

In addition to the application of 1% xylocaine noted above, using Allium sativum extract (or alliin/allicin or analogs) can enhance curcumin through the skin, such as for the treatment of deep injury such as keloidal scars and photoaging skin as well as for superficial arthritis. Using purified alliin/allicin or analogs to enhance curcumin penetration packaged in microspheres through target organ cell membranes, such as liver, brain, or joints, with potential use in cancer, Alzheimer's disease and arthritis is also possible utilizing the within described method and composition. If appropriately packaged and given intravenously, S-allyl molecules may also be used to enhance the penetration of non-lipid soluble molecules, including curcumin, through cell membranes in solid organs, such as the liver, pancreas, kidneys, uterus, prostrate, bone, joints and brain.

Similarly, Allium sativum extract (or alliin/allicin or analogs) can be used to enhance other non-lipid soluble medicaments through the skin for topical use, including anti-viral agents, such as acyclovir, valacyclovir and famciclovir; anti-fungal agents such as terbenafin, griseofulvin (anti-fungal), clotrimazole or ketoconazole; and topical antibiotics, such as clindamycin, neomycin, polysporin, bacitracin, etc.

Alliin/allicin or analogs can also be used in an inhaler to deliver non-lipid soluble medicaments to the sinuses or lungs as well as an aid for the delivery of non-lipid soluble medicaments to mucous membranes.

The illustrations and examples provided herein are for explanatory purposes and are not intended to limit the scope of the appended claims. This disclosure is to be considered an exemplification of the principles of the invention and is not intended to limit the spirit and scope of the invention and/or claims of the embodiment illustrated. Those skilled in the art will make modifications to the invention for particular applications of the invention.

What is claimed is:

1. A composition providing increased penetration of lipid insoluble medicaments through cell membranes comprising:
an S-Allyl-containing molecule; and
a lipid insoluble medicament.

2. The composition as defined in claim 1 wherein said composition is put into a gel medium.

3. The composition as defined in claim 1 wherein said S-Allyl-containing molecule is taken from the following group: alliin, allicin, Allium sativum extract, DMSO.

4. The composition as defined in claim 1 wherein said lipid insoluble medicament is taken from the following group: xylocaine, curcumin, acyclovir, valacyclovir, famciclovir, terbenafin, griseofulvin, clotrimazole, ketoconazole, clindamycin, neomycin, polysporin, bactricin.

5. The composition as defined in claim 1 wherein said composition is housed in an inhaler.

6. The composition as defined in claim 1 wherein said composition is applied directly to mucous membranes.

7. A method of penetrating cell membranes with lipid insoluble medicaments comprising the steps of:
preparing a composition comprising
an S-Allyl-containing molecule; and
a lipid insoluble medicament; and
applying said composition to said site of said cell membranes with a swab or other application device.

8. The method as defined in claim 7 wherein there is a further step of injecting said lipid insoluble medicament into said site of said cell membranes.

9. The method as defined in claim 7 wherein said composition is put into a gel medium.

10. The method as defined in claim 7 wherein said S-Allyl-containing molecule is taken from the following group: alliin, allicin, Allium sativum extract, DMSO.

11. The method as defined in claim 7 wherein said lipid insoluble medicament is taken from the following group: xylocaine, curcumin, acyclovir, valacyclovir, famciclovir, terbenafin, griseofulvin, clotrimazole, ketoconazole, clindamycin, neomycin, polysporin, bactricin.

12. A method of penetrating cell membranes with lipid insoluble medicaments comprising the steps of:
preparing a composition comprising
an S-Allyl-containing molecule; and
a lipid insoluble medicament; and
administering said composition through an inhaler.

13. The method as defined in claim 12 wherein said S-Allyl-containing molecule is taken from the following group: alliin, allicin, Allium sativum extract, DMSO.

14. The method as defined in claim 12 wherein said lipid insoluble medicament is taken from the following group: xylocaine, curcumin, acyclovir, valacyclovir, famciclovir, terbenafin, griseofulvin, clotrimazole, ketoconazole, clindamycin, neomycin, polysporin, bactricin.

15. A method of penetrating cell membranes with lipid insoluble medicaments comprising the steps of:
preparing a composition comprising
an S-Allyl-containing molecule; and
a lipid insoluble medicament; and
administering said composition directly to a mucous membrane.

16. The method as defined in claim 15 wherein said S-Allyl-containing molecule is taken from the following group: alliin, allicin, Allium sativum extract, DMSO.

17. The method as defined in claim 15 wherein said lipid insoluble medicament is taken from the following group: xylocaine, curcumin, acyclovir, valacyclovir, famciclovir, terbenafin, griseofulvin, clotrimazole, ketoconazole, clindamycin, neomycin, polysporin, bactricin.

18. A method of penetrating cell membranes with lipid insoluble medicaments comprising the steps of:
preparing a composition comprising:
an S-allyl-containing molecule; and
a lipid insoluble medicament; and
administering intravenously for the enhancement of the penetration of non-lipid soluble molecules through cell membranes in solid organs, such as the liver, pancreas, kidneys, uterus, prostrate, bone, joints and brain.

19. The method as defined in claim 18 wherein said S-Allyl-containing molecule is taken from the following group: alliin, allicin, Allium sativum extract, DMSO.

20. The method as defined in claim 18 wherein said lipid insoluble medicament is taken from the following group: xylocaine, curcumin, acyclovir, valacyclovir, famciclovir, terbenafin, griseofulvin, clotrimazole, ketoconazole, clindamycin, neomycin, polysporin, bactricin.
